(19) United States
(12) Patent Application Publication
(10) Pub. No.: US 2008/0312261 A1
(43) Pub. Date: Dec. 18, 2008

(54) 4- (4-Bromo-2-Fluoroanilino) -6- Methoxy-7- (1-Methylpiperidin-4-Ylmethoxy) Quinazoline Monohydrate

(76) Inventors: Rebecca Jane Booth, Cheshire (GB); Brian Roger Meyrick, Cheshire (GB); Zakariya Patel, Leicestershire (GB); Richard Anthony Storey, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/088,679

(22) PCT Filed: Sep. 27, 2006
(86) PCT No.: PCT/GB2006/003594

§ 371 Date: Aug. 28, 2008

(30) Foreign Application Priority Data
Sep. 30, 2005 (GB) 0519878.3

(51) Int. Cl. A61K 31/517; C07D 401/12; A61P 9/00

(52) U.S. Cl. 514/266.22; 544/293

(57) ABSTRACT

The present invention relates to a ZD6474 monohydrate, to processes for the preparation of a ZD6474 monohydrate, to pharmaceutical compositions comprising a ZD6474 monohydrate as the active ingredient, to the use of a ZD6474 monohydrate in the manufacture of medicaments for use in the production of antiangiogenic and/or vascular permeability reducing effects in warm-blooded animals such as humans, and to the use of a ZD6474 monohydrate in methods for the treatment of disease states associated with angiogenesis and/or increased vascular permeability, such as cancer, in warm-blooded animals such as humans.

The present invention relates to a novel form of ZD6474. More specifically, the present invention relates to a ZD6474 monohydrate, to processes for the preparation of a ZD6474 monohydrate, to pharmaceutical compositions comprising a ZD6474 monohydrate as the active ingredient, to the use of a ZD6474 monohydrate in the manufacture of medicaments for use in the production of antiangiogenic and/or vascular permeability reducing effects in warm-blooded animals such as humans, and to the use of a ZD6474 monohydrate in methods for the treatment of disease states associated with angiogenesis and/or increased vascular permeability, such as cancer, in warm-blooded animals such as humans.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified, including acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fins-like tyrosine kinase receptor, Flt-1, the kinase insert domain-containing receptor, KDR (also referred to as Flk-1), and another fins-like tyrosine kinase receptor, Flt-4. Two of these related RTJs, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington D.C.), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J., Orci, L. and Vassalli, J. D., Enzyme Protein, 49: 138-162, 1996). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Büttner, M., Rziha, H-J., and Dehio, C., EMBO J., 18: 363-374, 1999; Zeng, H., Sanyal, S, and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

Compounds which inhibit the effects of VEGF are of value in the treatment of disease states associated with angiogenesis and/or increased vascular permeability such as cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, lymphoedema, dysfunctional uterine bleeding and ocular diseases with retinal vessel proliferation including macular degeneration.

Quinazoline derivatives that are inhibitors of VEGF receptor tyrosine kinase are described in WO 98/13354 and WO 01/32651. In WO 98/13354 and WO 01/32651 compounds are described which possess activity against VEGF receptor tyrosine kinase (VEGF RTK) whilst possessing some activity against epidermal growth factor (EGF) receptor tyrosine kinase (EGF RTK).

ZD6474 is 4-(4-bromo-2-fluoroanilino)-6-methoxy-7-(1-methylpiperidin-4-ylmethoxy)quinazoline:

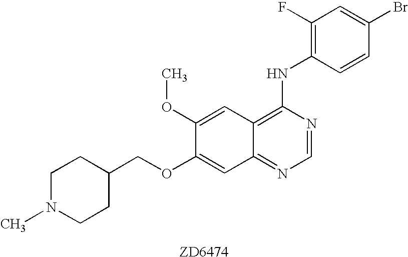

ZD6474 falls within the broad disclosure of WO 98/13354 and is exemplified in WO 01/32651. ZD6474 is a potent inhibitor of VEGF RTK and also has some activity against EGF RTK. ZD6474 has been shown to elicit broad-spectrum anti-tumour activity in a range of models following once-daily oral administration (Wedge S. R., Ogilvie D. J., Dukes M. et al, Proc. Am. Assoc. Canc. Res. 2001; 42: abstract 3126).

WO 01/32651 describes the preparation of ZD6474.

In Example 2a of WO 01/32651, the hydrochloride salt of ZD6474 is prepared and isolated.

In Example 2b of WO 01/32651, ZD6474 free base is prepared and isolated. During the isolation step, magnesium sulfate is used to dry the product. Elemental analysis of the isolated ZD6474 free base shows that it does not contain water. In other words, the isolated ZD6474 free base is in an anhydrous form.

In Example 2c of WO 01/32651, the hydrochloride salt of ZD6474 is prepared and isolated. In one aspect, the isolated hydrochloride salt of ZD6474 is dissolved in dimethylsulfoxide and converted to ZD6474 free base (in dimethylsulfoxide solution) by adding solid potassium carbonate. The ZD6474 free base in dimethylsulfoxide solution is in an anhydrous form. The ZD6474 free base in dimethylsulfoxide solution is then converted to the trifluoroacetate salt of ZD6474 by adding trifluoroacetic acid.

In another aspect of Example 2c of WO 01/32651, the ZD6474 free base is isolated as a solid. First, the isolated hydrochloride salt of ZD6474 is converted to ZD6474 free base by suspending the hydrochloride salt in methylene chloride and washing the suspension with saturated aqueous sodium hydrogen carbonate to provide a solution of ZD6474 free base in methylene chloride. The methylene chloride solution of ZD6474 free base is then dried using magnesium sulfate and the volatiles removed by evaporation. This procedure is repeated as Example 1 of the present application and provides the ZD6474 free base in crystalline, anhydrous form.

Thus, WO 01/32651 discloses both the hydrochloride salt of ZD6474 and ZD6474 free base. The ZD6474 free base that is obtained as a solid in the examples of WO 01/32651 is in an anhydrous form.

The processes described in WO 01/32651 for preparing the hydrochloride salt of ZD6474 and the anhydrous form of ZD6474 free base are also described and/or referenced in publications relating to combination therapies including ZD6474, such as WO 03/039551, WO 2004/014383, WO 2004/014426, WO 2004/032937, WO 2004/071397 and WO 2005/004870.

For the avoidance of doubt, the term “ZD6474” as used hereinafter refers to the ZD6474 free base, unless otherwise stated.

The anhydrous form of ZD6474 may be prepared using the processes described in WO 01/32651. An alternative process for preparing and isolating the anhydrous form of ZD6474 free base is described in Example 2 of the present application.

The anhydrous form of ZD6474 is a crystalline solid under ambient conditions. Differential Scanning Calorimetry (DSC) analysis was conducted on the anhydrous form of ZD6474 according to the method described hereinafter and shows a large, sharp endotherm with an onset temperature of between 230° C. and 240° C. due to melting (FIG. 1). It will be understood that the onset and/or peak temperature values of the DSC may vary slightly from one machine to another or from one sample to another, and so the values quoted are not to be construed as absolute.

Thermogravimetric (TGA) analysis was conducted on the anhydrous form of ZD6474 according to the method described hereinafter and shows no weight loss prior to melting (FIG. 1). This is indicative of the anhydrous form of ZD6474.

Karl Fischer analysis was conducted on the anhydrous form of ZD6474 according to the method described hereinafter and yields a figure of from 0.01 to 0.23% weight/weight. This is indicative of the anhydrous form of ZD6474.

The anhydrous form of ZD6474 is characterised in providing at least one of the following 2 theta values measured using CuKα radiation: 15.0° and 21.40. The anhydrous form of ZD6474 is characterised in providing a CuKα X-ray powder diffraction pattern as shown in FIG. 2. The ten most prominent peaks are shown in Table 1.

TABLE 1
Ten most prominent X-Ray Powder Diffraction
peaks for the anhydrous form of ZD6474

| Angle 2- | Intensity | Relative |
|---|---|---|
| Theta (° 2θ) | Count | Intensity |
| 15.0 | 100 | vs |
| 21.4 | 92.8 | vs |
| 23.3 | 63.7 | vs |
| 20.7 | 48.3 | vs |
| 18.9 | 40.4 | vs |
| 18.1 | 40.1 | vs |
| 23.7 | 39.2 | vs |
| 8.3 | 28.9 | vs |
| 22.1 | 25.9 | vs |
| 29.5 | 23.2 | s |

vs = very strong;
s = strong

Dynamic Vapour Sorption (DVS) analysis was carried out according to the method described hereinafter and shows that the anhydrous form of ZD6474 is non-hygroscopic (FIG. 3). At 95% relative humidity, the anhydrous form of ZD6474 absorbed only 0.63% weight/weight water, suggesting that there was no conversion to a hydrated form of ZD6474. The anhydrous form of ZD6474, therefore, is kinetically stable on the DVS timescale.

It is desirable to identify alternative stable forms of a pharmaceutically active compound. Alternative stable forms of a pharmaceutically active compound, for example alternative stable crystalline forms, are advantageous for formulation and processing on a commercial scale. For example, stable crystalline forms provide a low risk of conversion to another form during formulation procedures, which provides predictability of the properties of a final formulation.

The present invention is concerned with the identification of alternative forms of ZD6474, such as forms that are different to the anhydrous form of ZD6474 and that have improved solid-state properties in certain environments. For example, in one aspect, the present invention is concerned with the identification of alternative forms of ZD6474 that are especially useful in aqueous systems and/or in high humidity environments.

An example of an alternative form of ZD6474 is a hydrated form of ZD6474. In WO 01/32651 it says that the compounds it describes can exist in solvated as well as unsolvated forms such as, for example, hydrated forms.

Nowhere in WO 01/32651 does it state that a particular hydrate of a particular compound described therein will possess unexpected and/or beneficial properties.

We have now found that the monohydrate form of ZD6474 is an advantageously stable crystalline form of ZD6474 at ambient temperature and humidity. The crystalline monohydrate form of ZD6474 is especially suitable for use in aqueous environments, such as in aqueous suspension formulations, and/or in high humidity environments. Furthermore, the crystalline monohydrate form of ZD6474 is simple to process. For example, this form of ZD6474 may readily be dried on large scales (such as by fluid bed drying during formulation) at a temperature of about 30-40° C. without appreciable dehydration, it may undergo wet granulation without risk of hydration and it may be stored at a range of humidities. Additionally, processes for preparing the crystalline monohydrate form of ZD6474 also allow easy removal of particular water-soluble impurities.

According to the present invention there is provided a ZD6474 monohydrate. ZD6474 monohydrate is readily crystallised, is highly crystalline and is non-hygroscopic (by DVS measurements).

ZD6474 monohydrate in a crystalline form is characterised in providing at least one of the following 2 theta values measured using CuKα radiation: 10.8° and 21.0°. ZD6474 monohydrate in a crystalline form is characterised in providing an X-ray powder diffraction pattern, substantially as shown in FIG. 4. The ten most prominent peaks are shown in Table 2:

TABLE 2
Ten most prominent X-Ray Powder Diffraction
peaks for the monohydrate form of ZD6474

| Angle 2- | Intensity | Relative |
|---|---|---|
| Theta (° 2θ) | Count | Intensity |
| 10.8 | 100 | vs |
| 21.0 | 84.6 | vs |
| 18.4 | 63.5 | vs |
| 11.9 | 60.4 | vs |
| 18.9 | 40.4 | vs |
| 18.1 | 40.1 | vs |
| 22.1 | 51.1 | vs |
| 11.4 | 38.9 | vs |
| 20.1 | 38.7 | vs |
| 24.0 | 38.3 | vs |

vs = very strong

According to the present invention there is provided a ZD6474 monohydrate in a crystalline form, wherein the monohydrate has an X-ray powder diffraction pattern with at least one specific peak at about 2-theta=10.80.

According to the present invention there is provided a ZD6474 monohydrate in a crystalline form, wherein the monohydrate has an X-ray powder diffraction pattern with at least one specific peak at about 2-theta=21.00.

According to the present invention there is provided a ZD6474 monohydrate in a crystalline form, wherein the monohydrate has an X-ray powder diffraction pattern with at least two specific peaks at about 2-theta=10.8° and 21.0°.

According to the present invention there is provided a ZD6474 monohydrate in a crystalline form, wherein the monohydrate has an X-ray powder diffraction pattern with specific peaks at about 2-theta=10.8, 21.0, 18.4, 11.9, 18.9, 18.1, 22.1, 11.4, 20.1 and 24.0°.

According to the present invention there is provided a ZD6474 monohydrate in a crystalline form, wherein the monohydrate has an X-ray powder diffraction pattern substantially the same as the X-ray powder diffraction pattern shown in FIG. 4.

According to the present invention there is provided a ZD6474 monohydrate in a crystalline form, wherein the monohydrate has an X-ray powder diffraction pattern with at least one specific peak at 2-theta=10.80 plus or minus 0.5° 2-theta.

According to the present invention there is provided a ZD6474 monohydrate in a crystalline form, wherein the monohydrate has an X-ray powder diffraction pattern with at least one specific peak at 2-theta=21.0° plus or minus 0.5° 2-theta.

According to the present invention there is provided a ZD6474 monohydrate in a crystalline form, wherein the monohydrate has an X-ray powder diffraction pattern with at least two specific peaks at 2-theta=10.8° and 21.0° wherein said values may be plus or minus 0.5° 2-theta.

According to the present invention there is provided a ZD6474 monohydrate in a crystalline form, wherein the monohydrate has an X-ray powder diffraction pattern with specific peaks at 2-theta=10.8, 21.0, 18.4, 11.9, 18.9, 18.1, 22.1, 11.4, 20.1 and 24.0°, wherein said values may be plus or minus 0.5° 2-theta.

According to the present invention there is provided a ZD6474 monohydrate in a crystalline form, wherein the monohydrate has an X-ray powder diffraction pattern with at least one specific peak at 2-theta=10.8°.

According to the present invention there is provided a ZD6474 monohydrate in a crystalline form, wherein the monohydrate has an X-ray powder diffraction pattern with at least one specific peak at 2-theta=21.0°.

According to the present invention there is provided a ZD6474 monohydrate in a crystalline form, wherein the monohydrate has an X-ray powder diffraction pattern with at least two specific peaks at 2-theta=10.8° and 21.0°.

According to the present invention there is provided a ZD6474 monohydrate in a crystalline form, wherein the monohydrate has an X-ray powder diffraction pattern with specific peaks at 2-theta=10.8, 21.0, 18.4, 11.9, 18.9, 18.1, 22.1, 11.4, 20.1 and 24.0°.

According to the present invention there is provided a ZD6474 monohydrate in a crystalline form, wherein the monohydrate has an X-ray powder diffraction pattern as shown in FIG. 4.

In the preceding paragraphs defining the X-ray powder diffraction peaks for the ZD6474 monohydrate in a crystalline form, the term “at about” is used in the expression “ . . . at about 2-theta= . . . ” to indicate that the precise position of peaks (i.e. the recited 2-theta angle values) should not be construed as being absolute values because, as will be appreciated by those skilled in the art, the precise position of the peaks may vary slightly between one machine and another, from one sample to another, or as a result of slight variations in measurement conditions utilised. In one embodiment about 2-theta=10.8° would mean 2-theta=10.8±0.5°, in another embodiment 2-theta=10.8±0.2° and in a further embodiment 2-theta=10.8±0.1°. It is also stated in the preceding paragraphs that the ZD6474 monohydrate in a crystalline form provides an X-ray powder diffraction pattern “substantially” the same as the X-ray powder diffraction pattern shown in FIG. 4. It shall be appreciated that the use of the term “substantially” in this context is also intended to indicate that the 2-theta angle values of the X-ray powder diffraction patterns may vary slightly from one machine to another, from one sample to another, or as a result of slight variations in measurement conditions utilised, so the peak positions shown in the Figure are again not to be construed as absolute values.

DSC analysis (details given hereinafter) was conducted on ZD6474 monohydrate and shows a large broad endotherm with an onset temperature of between 50° C. and 120° C. due to dehydration (so as to produce the anhydrous form of ZD6474), as well as a large narrow endotherm with an onset temperature of between 230° C. and 240° C. due to melting of the anhydrous form of ZD6474 (FIG. 5).

TGA analysis (details given hereinafter) was conducted on ZD6474 monohydrate and shows a weight loss of about 3.7% between 69° C. and 111° C. (FIG. 5), which corresponds to the loss of the water of hydration from ZD6474 monohydrate. It will be understood that the temperature values of the TGA may vary slightly from one machine to another or from one sample to another, and so the values quoted are not to be construed as absolute.

Karl Fischer analysis (details given hereinafter) was conducted on ZD6474 monohydrate and yields a figure of about 3.9% suggesting that all the weight loss is due to water loss. As the skilled person would appreciate, the weight percentage of water in ZD6474 monohydrate is 3.65%.

Dynamic Vapour Sorption (DVS) analysis (details given hereinafter) was conducted on ZD6474 monohydrate and shows that ZD6474 monohydrate is non-hygroscopic (FIG. 6). The DVS analysis shows that the ZD6474 monohydrate substantially (less than 5%) does not convert to the anhydrous form of ZD6474 during drying at 25° C. and 0% relative humidity. A plot of the percentage weight change on storage of ZD6474 monohydrate at 0% relative humidity at 25° C. (FIG. 7) shows that once surface moisture has been removed, the rate of weight loss is extremely slow. A plot of the percentage weight change on storage of ZD6474 monohydrate at 0% relative humidity at 40° C. (FIG. 8) shows that the rate of weight loss is faster at this temperature but is still surprisingly slow for a hydrated compound in this environment. The ZD6474 monohydrate, therefore, is kinetically stable on the DVS timescale.

Slurry experiments were conducted as described in Example 3 of the present application (and as described in Zhu, H. J., Yuen, C., Grant, D. J. W., Int. J. Pharm., (1996) 135 (1,2) 151-160) to identify the conditions at which the ZD6474 monohydrate is the most stable crystalline form. These experiments show that at 25° C., the anhydrous form of ZD6474 is the thermodynamically stable form at less than or equal to 30% relative humidity. At 25° C., the ZD6474 monohydrate is the thermodynamically stable form at greater than or equal to 40% relative humidity. Therefore, at 25° C. and 50% relative humidity, the ZD6474 monohydrate is the most stable form.

When it is stated that the present invention relates to a crystalline form of ZD6474 monohydrate, the degree of crystallinity is conveniently greater than about 60%, more conveniently greater than about 80%, preferably greater than about 90% and more preferably greater than about 95%. Most preferably the degree of crystallinity is greater than about 98%.

For the avoidance of doubt, by the term “ambient conditions” we mean ambient temperature and humidity. By the term “ambient temperature” we mean a temperature in the range of from 15 to 30° C., particularly a temperature of about 25° C. By the term “ambient humidity” we mean between about 45 and 60% relative humidity. By the term “relative humidity” we mean the amount (%) of atmospheric moisture present relative to the amount that would be present if the air were saturated. As will be appreciated by those skilled in the art, relative humidity is a function of both moisture content and temperature.

The ZD6474 monohydrate crystalline form provides an X-ray powder diffraction pattern substantially the same as the X-ray powder diffraction pattern shown in FIG. 4 and has substantially the ten most prominent peaks (angle 2-theta values) shown in Table 2. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement variations depending on measurement conditions (such as equipment or machine used). In particular, it is generally known that intensities in an X-ray powder diffraction pattern may fluctuate depending on measurement conditions (for example preferred orientation). Therefore it should be understood that the ZD6474 monohydrate form of the present invention is not limited to the crystals that provide X-ray powder diffraction patterns identical to the X-ray powder diffraction pattern shown in FIG. 4, and any crystals providing X-ray powder diffraction patterns substantially the same as that shown in FIG. 4 fall within the scope of the present invention. A person skilled in the art of X-ray powder diffraction is able to judge the substantial identity of X-ray powder diffraction patterns.

Persons skilled in the art of X-ray powder diffraction will realise that the relative intensity of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is plus or minus 0.5° 2-theta or less, for example 0.2° 2-theta or ideally 0.1° 2-theta, and such degree of a measurement error should be taken into account when considering the X-ray powder diffraction patterns in FIGS. 2 and 4 and when reading Tables 1 and 2. Furthermore, it should be understood that intensities may fluctuate depending on experimental conditions and sample preparation (for example preferred orientation).

For the avoidance of doubt, the term “ZD6474 free base” refers to each and every form of ZD6474 free base, whereas “ZD6474 anhydrous” refers to the particular anhydrous form of ZD6474 free base and “ZD6474 monohydrate” refers to the particular monohydrate form of ZD6474 free base.

According to a further aspect of the invention there is provided a pharmaceutical composition which comprises a ZD6474 monohydrate as defined hereinbefore in association with a pharmaceutically acceptable excipient or carrier.

The composition may be in a form suitable for oral administration, (for example as tablets, lozenges, hard or soft capsules, aqueous or oily suspensions, emulsions, dispersible powders or granules, syrups or elixirs), for administration by inhalation (for example as a finely divided powder or a liquid aerosol), for administration by insufflation (for example as a finely divided powder), for parenteral injection (for example as a sterile solution, suspension or emulsion for intravenous, subcutaneous, intramuscular, intravascular or infusion dosing), for topical administration (for example as creams, ointments, gels, or aqueous or oily solutions or suspensions), or for rectal administration (for example as a suppository). Preferably ZD6474 monohydrate is administered orally. In general the above compositions may be prepared in a conventional manner using conventional excipients.

The compositions of the present invention are advantageously presented in unit dosage form. ZD6474 monohydrate will normally be administered to a warm-blooded animal at a unit dose within the range 10 to 500 mg per square metre body area of the animal, for example approximately 0.3 to 15 mg/kg in a human. A unit dose in the range, for example, 0.3 to 15 mg/kg, for example 0.5 to 5 mg/kg is envisaged and this is normally a therapeutically-effective dose. A unit dosage form such as a tablet or capsule will usually contain, for example 25 to 500 mg of active ingredient. Preferably a daily dose in the range of 0.5 to 5 mg/kg is employed. The size of the dose required for the therapeutic or prophylactic treatment of a particular disease state will necessarily be varied depending on the host treated, the route of administration and the severity of the illness being treated.

Accordingly the practitioner who is treating any particular patient may determine the optimum dosage.

According to a further aspect of the present invention there is provided a ZD6474 monohydrate as defined hereinbefore for use in a method of treatment of the human or animal body by therapy.

A further feature of the present invention is a ZD6474 monohydrate as defined hereinbefore for use as a medicament, conveniently a ZD6474 monohydrate as defined hereinbefore for use as a medicament for producing an antiangiogenic and/or vascular permeability reducing effect in a warm-blooded animal such as a human being.

Thus according to a further aspect of the invention there is provided the use of an ZD6474 monohydrate as defined hereinbefore in the manufacture of a medicament for use in the production of an antiangiogenic and/or vascular permeability reducing effect in a warm-blooded animal such as a human being.

According to a further feature of the invention there is provided a method for producing an antiangiogenic and/or vascular permeability reducing effect in a warm-blooded animal, such as a human being, in need of such treatment which comprises administering to said animal an effective amount of a ZD6474 monohydrate as defined hereinbefore.

ZD6474 monohydrate is an antiangiogenic and/or vascular permeability reducing agent and may be applied as a sole therapy or may involve, in addition to ZD6474 monohydrate, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to ZD6474 monohydrate may be: surgery, radiotherapy or chemotherapy. Such chemotherapy may cover three main categories of therapeutic agent:

(i) other antiangiogenic agents such as those which inhibit the effects of vascular endothelial growth factor, (for example the anti-vascular endothelial cell growth factor antibody bevacizumab [Avastin™], and those that work by different mechanisms from those defined hereinbefore (for example linomide, inhibitors of integrin αvβ3 function, angiostatin, razoxin, thalidomide), and including vascular targeting agents (for example combretastatin phosphate and compounds disclosed in International Patent Applications WO 00/40529, WO 00/41669, WO 01/92224, WO 02/04434 and WO 02/08213 and the vascular damaging agents described in International Patent Application WO 99/02166 the entire disclosure of which document is incorporated herein by reference, (for example N-acetylcolchinol-O-phosphate));
(ii) cytostatic agents such as antioestrogens (for example tamoxifen, toremifene, raloxifene, droloxifene, iodoxyfene), oestrogen receptor down regulators (for example fulvestrant), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide, buserelin), inhibitors of 5α-reductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor), such inhibitors include growth factor antibodies, growth factor receptor antibodies, (for example the anti-erbb2 antibody trastuzumab [Herceptin™] and the anti-erbb1 antibody cetuximab [C225]), farnesyl transferase inhibitors, tyrosine kinase inhibitors for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine(gefitinib, ZD 1839), N-(3-ethynylphenyl)-6,7-bis(2-methoxyethoxy)quinazolin-4-amine(erlotinib, OSI-774) and 6-acrylamido-N-(3-chloro-4-fluorophenyl)-7-(3-morpholinopropoxy)quinazolin-4-amine (CI 1033)) and serine/threonine kinase inhibitors); and
(iii) antiproliferative/antineoplastic drugs and combinations thereof, as used in medical oncology, such as antimetabolites (for example antifolates like methotrexate, fluoropyrimidines like 5-fluorouracil, tegafur, purine and adenosine analogues, cytosine arabinoside); antitumour antibiotics (for example anthracyclines like adriamycin, bleomycin, doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine, vinblastine, vindesine, vinorelbine, and taxoids like taxol, taxotere); topoisomerase inhibitors (for example epipodophyllotoxins like etoposide and teniposide, amsacrine, topotecan, camptothecin and also irinotecan); also enzymes (for example asparaginase); and thymidylate synthase inhibitors (for example raltitrexed); and additional types of chemotherapeutic agent include:
(iv) biological response modifiers (for example interferon);
(v) antibodies (for example edrecolomab);
(vi) antisense therapies, for example those which are directed to the targets listed above, such as ISIS 2503, an anti-ras antisense;
(vii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(viii) immunotherapy approaches, including for example ex-vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

For example such conjoint treatment may be achieved by way of the simultaneous, sequential or separate administration of a ZD6474 monohydrate as defined hereinbefore and a vascular targeting agent described in WO 99/02166 such as N-acetylcolchinol-O-phosphate (Example 1 of WO 99/02166).

It is known from WO 01/74360 that antiangiogenics can be combined with antihypertensives. A ZD6474 monohydrate of the present invention can also be administered in combination with an antihypertensive. An antihypertensive is an agent that lowers blood pressure (see, for example, WO 01/74360 which is incorporated herein by reference).

Thus according to the present invention there is provided a method of treatment of a disease state associated with angiogenesis which comprises the administration of an effective amount of a combination of a ZD6474 monohydrate as defined hereinbefore and an anti-hypertensive agent to a warm-blooded animal, such as a human being.

According to a further feature of the present invention there is provided the use of a combination of a ZD6474 monohydrate as defined hereinbefore and an anti-hypertensive agent for use in the manufacture of a medicament for the treatment of a disease state associated with angiogenesis in a warm-blooded mammal, such as a human being.

According to a further feature of the present invention there is provided a pharmaceutical composition comprising a ZD6474 monohydrate as defined hereinbefore and an anti-hypertensive agent for the treatment of a disease state associated with angiogenesis in a warm-blooded mammal, such as a human being.

According to a further aspect of the present invention there is provided a method for producing an anti-angiogenic and/or vascular permeability reducing effect in a warm-blooded animal, such as a human being, which comprises administering to said animal an effective amount of a ZD6474 monohydrate as defined hereinbefore and an anti-hypertensive agent.

According to a further aspect of the present invention there is provided the use of a combination of a ZD6474 monohydrate as defined hereinbefore and an anti-hypertensive agent for the manufacture of a medicament for producing an anti-angiogenic and/or vascular permeability reducing effect in a warm-blooded mammal, such as a human being.

Preferred antihypertensive agents are calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers α-blockers). Particular antihypertensive agents are calcium channel blockers, angiotensin converting enzyme inhibitors (ACE inhibitors), angiotensin II receptor antagonists (A-II antagonists) and beta-adrenergic receptor blockers (β-blockers), especially calcium channel blockers.

As stated above ZD6474 monohydrate is of interest for its antiangiogenic and/or vascular permeability reducing effects. ZD6474 monohydrate is expected to be useful in a wide range of disease states including cancer, diabetes, psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation including age-related macular degeneration. Cancer may affect any tissue and includes leukaemia, multiple myeloma and lymphoma. In particular such compounds of the invention are expected to slow advantageously the growth of primary and recurrent solid tumours of, for example, the colon, breast, prostate, lungs and skin. More particularly such compounds of the invention are expected to inhibit any form of cancer associated with VEGF including leukaemia, multiple myeloma and lymphoma and also, for example, the growth of those primary and recurrent solid tumours which are associated with VEGF, especially those tumours which are significantly dependent on VEGF for their growth and spread, including for example, certain tumours of the colon, breast, prostate, lung, brain vulva and skin.

In addition to its use in therapeutic medicine, the ZD6474 monohydrate defined hereinbefore is also useful as pharmacological tools in the development and standardisation of in vitro and in vivo test systems for the evaluation of the effects of inhibitors of VEGF receptor tyrosine kinase activity in laboratory animals such as cats, dogs, rabbits, monkeys, rats and mice, as part of the search for new therapeutic agents.

The assays written up in WO 01/32651 and used to test ZD6474 are as follows:

### (a) In Vitro Receptor Tyrosine Kinase Inhibition Test

This assay determines the ability of a test compound to inhibit tyrosine kinase activity. DNA encoding VEGF or epidermal growth factor (EGF) receptor cytoplasmic domains may be obtained by total gene synthesis (Edwards M, International Biotechnology Lab 5(3), 19-25, 1987) or by cloning. These may then be expressed in a suitable expression system to obtain polypeptide with tyrosine kinase activity. For example VEGF and EGF receptor cytoplasmic domains, which were obtained by expression of recombinant protein in insect cells, were found to display intrinsic tyrosine kinase activity. In the case of the VEGF receptor Flt (Genbank accession number X51602), a 1.7 kb DNA fragment encoding most of the cytoplasmic domain, commencing with methionine 783 and including the termination codon, described by Shibuya et al (Oncogene, 1990, 5: 519-524), was isolated from cDNA and cloned into a baculovirus transplacement vector (for example pAcYM1 (see The Baculovirus Expression System: A Laboratory Guide, L. A. King and R. D. Possee, Chapman and Hall, 1992) or pAc360 or pBlueBacHis (available from Invitrogen Corporation)). This recombinant construct was co-transfected into insect cells (for example Spodoptera frugiperda 21(Sf21)) with viral DNA (eg Pharmingen BaculoGold) to prepare recombinant baculovirus. (Details of the methods for the assembly of recombinant DNA molecules and the preparation and use of recombinant baculovirus can be found in standard texts for example Sambrook et al, 1989, Molecular cloning—A Laboratory Manual, 2nd edition, Cold Spring Harbour Laboratory Press and O'Reilly et al, 1992, Baculovirus Expression Vectors—A Laboratory Manual, W.H. Freeman and Co, New York). For other tyrosine kinases for use in assays, cytoplasmic fragments starting from methionine 806 (KDR, Genbank accession number L04947) and methionine 668 (EGF receptor, Genbank accession number X00588) may be cloned and expressed in a similar manner.

For expression of cFlt tyrosine kinase activity, Sf21 cells were infected with plaque-pure cFlt recombinant virus at a multiplicity of infection of 3 and harvested 48 hours later. Harvested cells were washed with ice cold phosphate buffered saline solution (PBS) (10 mM sodium phosphate pH 7.4, 138 mM sodium chloride, 2.7 mM potassium chloride) then resuspended in ice cold HNTG/PMSF (20 mM Hepes pH 7.5, 150 mM sodium chloride, 10% v/v glycerol, 1% v/v Triton X100, 1.5 mM magnesium chloride, 1 mM ethylene glycol-bis(paminoethyl ether) N,N,N′,N′-tetraacetic acid (EGTA), 1 mM PMSF (phenylmethylsulphonyl fluoride); the PMSF is added just before use from a freshly-prepared 100 mM solution in methanol) using 1 ml HNTG/PMSF per 10 million cells. The suspension was centrifuged for 10 minutes at 13,000 rpm at 4° C., the supernatant (enzyme stock) was removed and stored in aliquots at −70° C. Each new batch of stock enzyme was titrated in the assay by dilution with enzyme diluent (100 mM Hepes pH 7.4, 0.2 mM sodium orthovanadate, 0.1% v/v Triton X100, 0.2 mM dithiothreitol). For a typical batch, stock enzyme is diluted 1 in 2000 with enzyme diluent and 50 μl of dilute enzyme is used for each assay well.

A stock of substrate solution was prepared from a random copolymer containing tyrosine, for example Poly (Glu, Ala, Tyr) 6:3:1 (Sigma P3899), stored as 1 mg/ml stock in PBS at −20° C. and diluted 1 in 500 with PBS for plate coating.

On the day before the assay 100 μl of diluted substrate solution was dispensed into all wells of assay plates (Nunc maxisorp 96-well immunoplates) which were sealed and left overnight at 4° C.

On the day of the assay the substrate solution was discarded and the assay plate wells were washed once with PBST (PBS containing 0.05% v/v Tween 20) and once with 50 mM Hepes pH 7.4.

Test compounds were diluted with 10% dimethylsulphoxide (DMSO) and 25 μl of diluted compound was transferred to wells in the washed assay plates. “Total” control wells contained 10% DMSO instead of compound. Twenty five microlitres of 40 mM manganese(II) chloride containing 8 μM adenosine-5′-triphosphate (ATP) was added to all test wells except “blank” control wells which contained manganese(II) chloride without ATP. To start the reactions 50 μl of freshly diluted enzyme was added to each well and the plates were incubated at room temperature for 20 minutes. The liquid was then discarded and the wells were washed twice with PBST. One hundred microlitres of mouse IgG anti-phosphotyrosine antibody (Upstate Biotechnology Inc. product 05-321), diluted 1 in 6000 with PBST containing 0.5% w/v bovine serum albumin (BSA), was added to each well and the plates were incubated for 1 hour at room temperature before discarding the liquid and washing the wells twice with PBST. One hundred microlitres of horse radish peroxidase (HRP)-linked sheep anti-mouse Ig antibody (Amersham product NXA 931), diluted 1 in 500 with PBST containing 0.5% w/v BSA, was added and the plates were incubated for 1 hour at room temperature before discarding the liquid and washing the wells twice with PBST. One hundred microlitres of 2,2′-azino-bis(3-ethylbenzthiazoline-6-sulphonic acid) (ABTS) solution, freshly prepared using one 50 mg ABTS tablet (Boehringer 1204 521) in 50 ml freshly prepared 50 mM phosphate-citrate buffer pH 5.0+0.03% sodium perborate (made with 1 phosphate citrate buffer with sodium perborate (PCSB) capsule (Sigma P4922) per 100 ml distilled water), was added to each well. Plates were then incubated for 20-60 minutes at room temperature until the optical density value of the “total” control wells, measured at 405 nm using a plate reading spectrophotometer, was approximately 1.0. “Blank” (no ATP) and “total” (no compound) control values were used to determine the dilution range of test compound which gave 50% inhibition of enzyme activity.

### (b) In Vitro HUVEC Proliferation Assay

This assay determines the ability of a test compound to inhibit the growth factor-stimulated proliferation of human umbilical vein endothelial cells (HUVEC).

HUVEC cells were isolated in MCDB 131 (Gibco BRL)+7.5% v/v foetal calf serum (FCS) and were plated out (at passage 2 to 8), in MCDB 131+2% v/v FCS+3 ηg/ml heparin+1 μg/ml hydrocortisone, at a concentration of 1000 cells/well in 96 well plates. After a minimum of 4 hours they were dosed with the appropriate growth factor (i.e. VEGF 3 ng/ml, EGF 3 ng/ml or b-FGF 0.3 ng/ml) and compound. The cultures were then incubated for 4 days at 37° C. with 7.5% carbon dioxide. On day 4 the cultures were pulsed with 1 μCi/well of tritiated-thymidine (Amersham product TRA 61) and incubated for 4 hours. The cells were harvested using a 96-well plate harvester (Tomtek) and then assayed for incorporation of tritium with a Beta plate counter. Incorporation of radioactivity into cells, expressed as cpm, was used to measure inhibition of growth factor-stimulated cell proliferation by compounds.

### (c) In Vivo Solid Tumour Disease Model

This test measures the capacity of compounds to inhibit solid tumour growth.

CaLu-6 tumour xenografts were established in the flank of female athymic Swiss nu/nu mice, by subcutaneous injection of 1×10⁶ CaLu-6 cells/mouse in 100 μl of a 50% (v/v) solution of Matrigel in serum free culture medium. Ten days after cellular implant, mice were allocated to groups of 8-10, so as to achieve comparable group mean volumes. Tumours were measured using vernier calipers and volumes were calculated as: (1×w)×√(1×w)×(π/6), where 1 is the longest diameter and w the diameter perpendicular to the longest diameter. Test compounds were administered orally once daily for a minimum of 21 days, and control animals received compound diluent. Tumours were measured twice weekly. The level of growth inhibition was calculated by comparison of the mean tumour volume of the control group versus the treatment group, and statistical significance determined using a Students' t-test and/or a Mann-Whitney Rank Sum Test. The inhibitory effect of compound treatment was considered significant when p<0.05.

The toxicological profile of compounds of the present invention may be assessed, for example using a rat 14 day study as described hereinafter.

### (d) 14 Day Toxicity Test in Rat

This test measures the activity of compounds in increasing the zone of hypertrophy in the femoral epiphyseal growth plates of the distal femur and proximal tibia, and allows assessment of histopathological changes in other tissues.

Angiogenesis is an essential event in endochondral ossification during long bone elongation, and vascular invasion of the growth plate has been suggested to depend upon VEGF production by hypertrophic chondrocytes. Expansion of the hypertrophic chondrocyte zone and inhibition of angiogenesis has been demonstrated following treatment with agents which specifically sequester VEGF, such as, for example, (i) a soluble VEGF receptor chimeric protein (Flt-(1-3)-IgG) in mice (Gerber, H-P., Vu, T. H., Ryan, A. M., Kowalski, J., Werb, Z. and Ferrara, N. VEGF couples hypertrophic cartilage remodelling, ossification and angiogenesis during endochondral bone formation, Nature Med., 5: 623-628, 1999) and (ii) a recombinant humanised anti-VEGF monoclonal IgG1 antibody in cynomologus monkey (Ryan, A. M., Eppler, D. B., Hagler, K. E., Bruner, R. H., Thomford, P. J., Hall, R. L., Shopp, G. M. and O'Niell, C. A. Preclinical Safety Evaluation of rhuMAbVEGF, an antiangiogenic humanised monoclonal antibody, Tox. Path., 27: 78-86, 1999).

An inhibitor of VEGF receptor tyrosine kinase activity should therefore also inhibit vascular invasion of cartilage, and increase the zone of hypertrophy in the femoral epiphyseal growth plates of the distal femur and proximal tibia in growing animals.

Compounds were initially formulated by suspension in a 1% (v/v) solution of polyoxyethylene (20) sorbitan mono-oleate in deionised water, by ball-milling at 4° C. overnight (at least 15 hours). Compounds were re-suspended by agitation immediately prior to dosing. Young Alderley Park rats (Wistar derived, 135-150 g in weight, 4 to 8 weeks of age, 5-6 per group) were dosed once-daily by oral gavage for 14 consecutive days with compound (at 0.25 ml/100 g body weight) or vehicle. On day 15 animals were humanely terminated using a rising concentration of carbon dioxide, and a post-mortem performed. A range of tissues, which included femoro-tibial joints, were collected and processed by standard histological techniques to produce paraffin wax sections. Histological sections were stained with haematoxylin and eosin and examined by light microscopy for histopathology. The femoral epiphyseal growth plate areas of the distal femur and proximal tibia were measured in sections of femur and tibia using morphometric image analysis. The increase in the zone of hypertrophy was determined by comparison of the mean epiphyseal growth plate area of the control group versus the treatment group, and statistical significance determined using a one-tailed Students' t-test. The inhibitory effect of compound treatment was considered significant when p<0.05.

As disclosed in WO 01/32651, ZD6474 (prepared according to the procedure described in Example 2 of WO 01/32651) tested according to (a), (b), (c) and (d) above gave the following results:

### (a) Flt—IC₅₀ of 1.6 μM

KDR—IC₅₀ of 0.04 μM

EGFR—IC₅₀ of 0.5 μM

### (b) VEGF—IC₅₀ of 0.06 μM

EGF—IC₅₀ of 0.17 μM

Basal —IC₅₀ of >3 μM

(c) 78% inhibition of tumour growth at 50 mg/kg; p<0.001 (Mann-Whitney Rank Sum Test);
(d) 75% increase in epiphyseal growth plate hypertrophy at 100 mg/kg/day in female rats; p<0.001 (one-tailed Students' t-test).

A ZD6474 monohydrate as defined hereinbefore may be prepared by any process known to be applicable to the preparation of chemically-related compounds. Such processes are provided as a further feature of the invention and are as described hereinafter. Necessary starting materials may be obtained by standard procedures of organic chemistry. The anhydrous form of ZD6474 may be prepared according to any of the processes described in WO 01/32651, see in particular Examples 2b and 2c of WO 01/32651. Alternatively necessary starting materials are obtainable by analogous procedures to those illustrated which are within the ordinary skill of an organic chemist.

The following process constitutes a further feature of the present invention.

### Synthesis of ZD6474 Monohydrate

(a) Such a process provides a further aspect of the present invention and comprises, for example, the steps of:
(i) dissolving ZD6474 free base in an aqueous organic solvent mixture to form a solution;
(ii) allowing spontaneous crystallisation to occur; and
(iii) isolating the crystalline solid so formed.
   (b) Another such process provides a further aspect of the present invention and comprises, for example, the steps of:
(i) dissolving ZD6474 free base in an aqueous organic solvent mixture to form a solution;
(ii) adding a seed of ZD6474 monohydrate to initiate crystallisation of ZD6474 monohydrate; and
(iii) isolating the crystalline solid so formed.

For part (i) of (a) and (b) above, the organic solvent used may be any non-solvating solvent. By the term “non-solvating solvent” we mean a solvent that does not form crystalline solvates with ZD6474. More specifically, the organic solvent includes water in an amount so as to provide a water activity of from about 0.4 to 1.0, especially of from about 0.5 to 0.95. By the term “water activity” we mean the available water in a substrate (for example a solvent) as a decimal fraction of the amount present when the substrate is in equilibrium with the surrounding atmosphere at a particular relative humidity. In other words, an equilibrium relative humidity of 70% around the substrate means that the substrate has a water activity of 0.70. For example, for part (i) of (a) and (b) above, the organic solvent may be an ether such as tetrahydrofuran. In particular, the tetrahydrofuran may contain from 5 to 10% (by volume), particularly 10%, water to provide the aqueous organic solvent mixture. In other words, the mixture may contain from 95 to 90% (by volume), particularly 90%, tetrahydrofuran and from 5 to 10% (by volume), particularly 10%, water.

For part (i) of (a) and (b) the mixture may, if required, be heated to reflux until dissolution has occurred. Alternatively, the mixture may, for example, be heated to a temperature less than the reflux temperature of the solvent mixture provided that dissolution of substantially all of the solid material has occurred. It will be appreciated that small quantities of insoluble material may be removed by filtration of the warmed mixture.

In (a) and (b) above the crystalline solid so formed may be isolated by any conventional method, for example by filtration. The isolated crystalline solid may then be dried. For example, when the crystalline solid is dried without humidification, a suitable drying temperature is from about 20 to 30° C., especially about 25° C. When the crystalline solid is dried with humidification, the drying temperature is from about 30 to 50° C., especially about 40° C.

The invention is illustrated hereinafter by means of the following non-limiting Examples, Data and Figures in which, unless otherwise stated:—

(i) evaporations were carried out by rotary evaporation in vacuo and work-up procedures were carried out after removal of residual solids such as drying agents by filtration;

(ii) yields are given for illustration only and are not necessarily the maximum attainable;

(iii) melting points are uncorrected and were determined using a Mettler DSC820e;

(iv) the structures of the end-products were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet; all samples run on a Bruker DPX 400 MHz at 300K in the solvent indicated, 16 scans, pulse repetition time 10 seconds;

(v) intermediates were not generally fully characterised and purity was assessed by NMR analysis; and

(vi) the following abbreviations have been used:—
RH relative humidity
THF tetrahydrofuran
IPA isopropanol
DMSO dimethylsulfoxide
DSC Differential Scanning Calorimetry
TGA Thermogravimetric Analysis
v/v volume/volume ratio
w/w weight/weight ratio

### EXAMPLE 1

### Repeat of the Isolation Step of ZD6474 Free Base of Example 2c of WO 01/32651

As discussed above, in Example 2c of WO 01/32651, ZD6474 free base is isolated as a solid. In Example 2c of WO 01/32651, the hydrochloride salt of ZD6474 is converted to ZD6474 free base by suspending the hydrochloride salt in methylene chloride and washing the suspension with saturated aqueous sodium hydrogen carbonate to provide a solution of ZD6474 free base in methylene chloride. The methylene chloride solution of ZD6474 free base is then dried using magnesium sulfate and the volatiles removed by evaporation.

In this example of the present application, the isolation step of Example 2c of WO 01/32651 was repeated from the step whereby a solution of ZD6474 free base in methylene chloride has been provided (which is washed with water). As a person skilled in the art would appreciate, the steps used prior to the isolation step to prepare the solution of ZD6474 free base in methylene chloride are irrelevant to the form of ZD6474 that is provided by means of the particular isolation step. Additionally, any neutralisation step(s) has no effect on the form of ZD6474 that is provided.

A sample of ZD6474 (250.5 mg) was placed in a Wheaton disposable glass scintillation vial and dichloromethane (10 ml) was added. The vial was capped and the mixture was swirled gently for 10 minutes to dissolve the solid. Water (5 ml) was then added to the solution and the mixture was shaken vigorously for 30 seconds. The mixture was allowed to stand for 2 minutes and then the dichloromethane layer was removed with a glass pipette and placed in another glass scintillation vial. Magnesium sulfate was added to the solution and the mixture was swirled to fully disperse the solid. The addition of magnesium sulfate was continued until the solid no longer clumped together but formed a fine dispersion on swirling. The mixture was allowed to stand overnight. The magnesium sulfate was then removed by filtration and rinsed with dichloromethane (1 ml). The filtrate and the washings were combined and allowed to evaporate to give a fine white crystalline solid. This material was then analysed by XRPD (according to the method described hereinafter). The XRPD trace (FIG. 9) shows that the material is the anhydrous form of ZD6474 (see FIG. 2). As the skilled person would appreciate, any differences in peak height are due to preferred crystal orientation.

### EXAMPLE 2

### Preparation of ZD6474 Anhydrous

ZD6474 free base was prepared according to the procedure described in Example 2b of WO 01/32651. The ZD6474 free base (10 g) was suspended in tetrahydrofuran (50 ml), water (25 ml) and n-butyl acetate (40 ml) and the suspension heated to reflux to give a solution. The aqueous phase was separated and the organic phase was filtered and washed with tetrahydrofuran (5 ml). n-Butyl acetate (60 ml) was added and the mixture distilled at atmospheric pressure until a contents temperature of 106° C. was achieved. The resulting slurry of ZD6474 was cooled and the solid isolated by filtration, washed with ethyl acetate (20 ml) and dried to provide ZD6474 anhydrous (9.2 g, 92%); NMR spectrum (pyridine-d5) 1.49 (2H, m), 1.75-1.90 (5H, m), 2.15 (3H, s), 2.76 (2H, m), 3.63 (3H, s), 3.97 (2H, d), 7.38 (1H, ddd), 7.49 (1H, dd), 7.64 (1H, s), 7.88 (1H, t), 7.89 (1H, s), 9.01 (1H, s), 10.37 (1H, s); Mass spectrum MH⁺ 475.

### EXAMPLE 3

### Slurry Experiments in Aqueous Isopropanol at Specific Water Activities to Investigate the Most Stable form of ZD6474 at Different Relative Humidities

ZD6474 anhydrous (50 mg) and ZD6474 monohydrate (50 mg) were slurried in different ratios of isopropanol/water having water activities of 0.3, 0.4 and 0.5 (corresponding to 30% relative humidity, 40% relative humidity and 50% relative humidity respectively) for 24 hours at 25° C. The resulting material was then filtered off and air-dried. These experiments indicated that at 25° C., ZD6474 anhydrous is the thermodynamically stable form at <30% relative humidity and ZD6474 monohydrate is the thermodynamically stable form at >40% relative humidity.

### EXAMPLE 4

### Preparation of ZD6474 Monohydrate

ZD6474 free base was prepared according to the procedure described in Example 2b of WO 01/32651. The ZD6474 free base (10.06 g) was added to aqueous tetrahydrofuran (90% tetrahydrofuran/10% water, volume/volume) at ambient temperature. The mixture was stirred and warmed to 40° C. until all solid had dissolved. Further ZD6474 free base (1.44 g) was added to the mixture at 42° C. and the mixture was stirred for 20 minutes to provide a clear solution. The solution was warmed to 50° C. and stirred at this temperature for 4 hours. The solution was then cooled to room temperature and stirred for 12 days to provide a slurry. The resulting solid was filtered under vacuum (600 to 700 mbar) and dried in air under vacuum (200 mbar) for 1 hour. Karl Fischer analysis was conducted on the dried ZD6474 product according to the method described hereinafter and yielded a figure of 3.904%, which is consistent with ZD6474 monohydrate; NMR spectrum (pyridine-d5) 1.49 (2H, m), 1.75-1.90 (5H, m), 2.15 (3H, s), 2.76 (2H, m), 3.63 (3H, s), 3.97 (2H, d), 7.38 (1H, ddd), 7.49 (1H, dd), 7.64 (1H, s), 7.88 (1H, t), 7.89 (1H, s), 9.01 (1H, s), 10.37 (1H, s); Mass spectrum MH⁺ 475.

### EXAMPLE 5

### Alternative Preparation Method of ZD6474 Monohydrate

This was prepared in a temperature controlled glass reaction vessel set at 30° C. The vessel was charged with anhydrous ZD6474. To this was added 3 relative volumes of tetrahydrofuran (stabilised) and 7 relative volumes of purified water (i.e. 3 litres of THF and 7 litres of water would be used for 1 kg of ZD6474). The contents were agitated to form a cream coloured slurry. The reaction turnover was typically complete in under an hour, but a small sample of the slurry can be taken after an hour, filtered, then a powder XRD spectrum taken to confirm this. The solid was isolated by filtering on a split Buchner funnel. The reaction vessel was washed with 2 relative volumes of water. The reaction vessel wash was then used as a displacement wash of the filter cake in the Buchner funnel. A further wash was performed using an additional 2 relative volumes of water added to the reaction vessel which was again used to wash the filter cake.

The solid was transferred to a vacuum oven and dried at ambient temperature until dry. During drying the solid was slurried regularly. Drying was very slow, typically a 350 g batch takes approximately 2 weeks to dry.

### EXAMPLE 6

### Alternative Preparation Method of ZD6474 Monohydrate

ZD6474 free base is prepared according to the procedure described in Example 2b of WO 01/32651. The ZD6474 free base (10.06 g) is added to aqueous tetrahydrofuran (90% tetrahydrofuran/10% water, volume/volume) at ambient temperature in a temperature controlled glass reaction vessel. The mixture is stirred and warmed to 40° C. until all solid is dissolved. Further ZD6474 free base (1.44 g) is added to the mixture at 42° C. and the mixture is stirred for 20 minutes to provide a clear solution. Optionally the solution is screened at this point. The solution is then warmed to 50° C. and is stirred at this temperature for 4 hours. The solution is then cooled to room temperature and is stirred for 1 day to provide a slurry. Then a small sample of the slurry is taken filtered, and a powder XRD spectra obtained. If the XRD spectrum shows all the anhydrous ZD6474 has been converted to the monohydrate this is isolated as detailed below. If the XRD spectrum shows a mixture of anhydrous ZD6474 and ZD6474 monohydrate, then the solution is agitated for a further 4 hours at ambient temperature, ideally about 20° C. and then is retested. If the XRD spectrum shows predominantly all anhydrous ZD6474 then a seed crystal of ZD6474 monohydrate (0.1-1% by weight of the theoretical final yield) and the solution is agitated for a further 4 hours at ambient temperature, ideally about 20° C., and then can be retested. This process can be repeated until all the anhydrous ZD6474 is converted to ZD6474 monohydrate.

The solid is isolated by filtering on a split buchner funnel. The reaction vessel is washed with 2 relative volumes of water. The reaction vessel wash is then used as a displacement wash of the filter cake in the buchner funnel. A further wash is performed using an additional 2 relative volumes of water added to the reaction vessel which is again used to wash the filter cake.

The solid is transferred to a vacuum oven and is dried at ambient temperature until dry. During drying the solid is slurried regularly. Drying is very slow, typically a 350 g batch takes approximately 2 weeks to dry.

### BRIEF DESCRIPTION OF THE DRAWINGS

FIG. 1: DSC and TGA Thermograms for ZD6474 anhydrous—with temperature in ° C. plotted on the horizontal axis and heat flow/% weight loss on the vertical axis. The top plot is the TGA plot and the lower plot is the DSC plot. The scale on the y axis for the TGA plot is 2 mg as indicated on the graph and the scale on the y axis for the DSC plot is 10 mW as indicated on the graph.

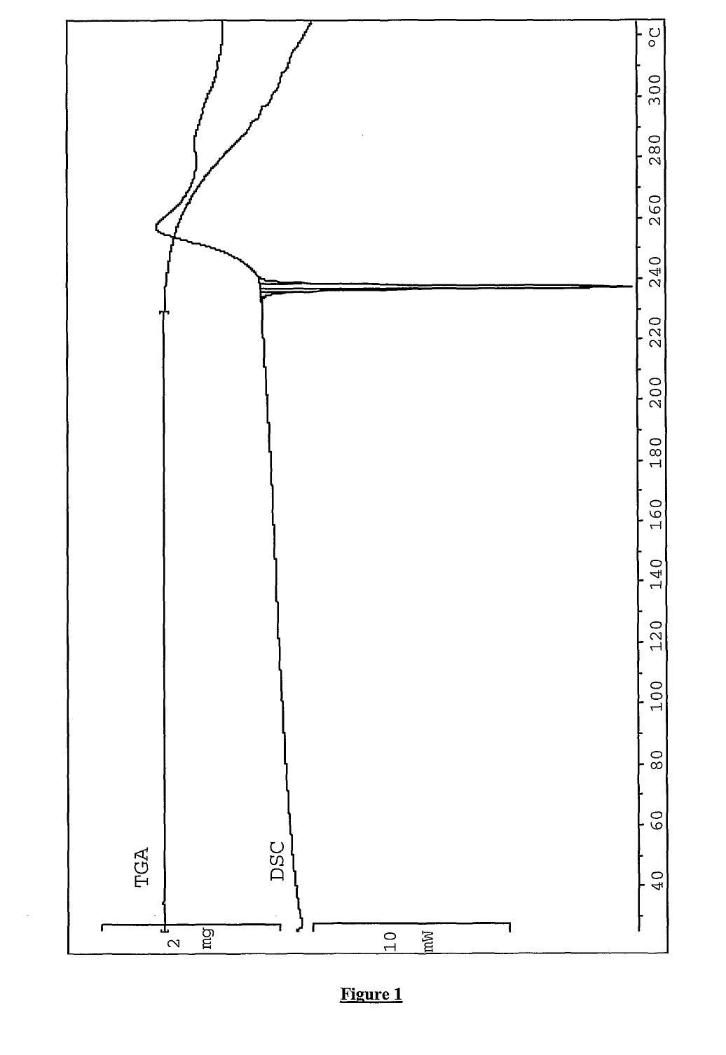

FIG. 2: X-Ray Powder Diffraction Pattern for ZD6474 anhydrous—with the 2 theta values plotted on the horizontal axis and the relative line intensity (counts) plotted on the vertical axis.

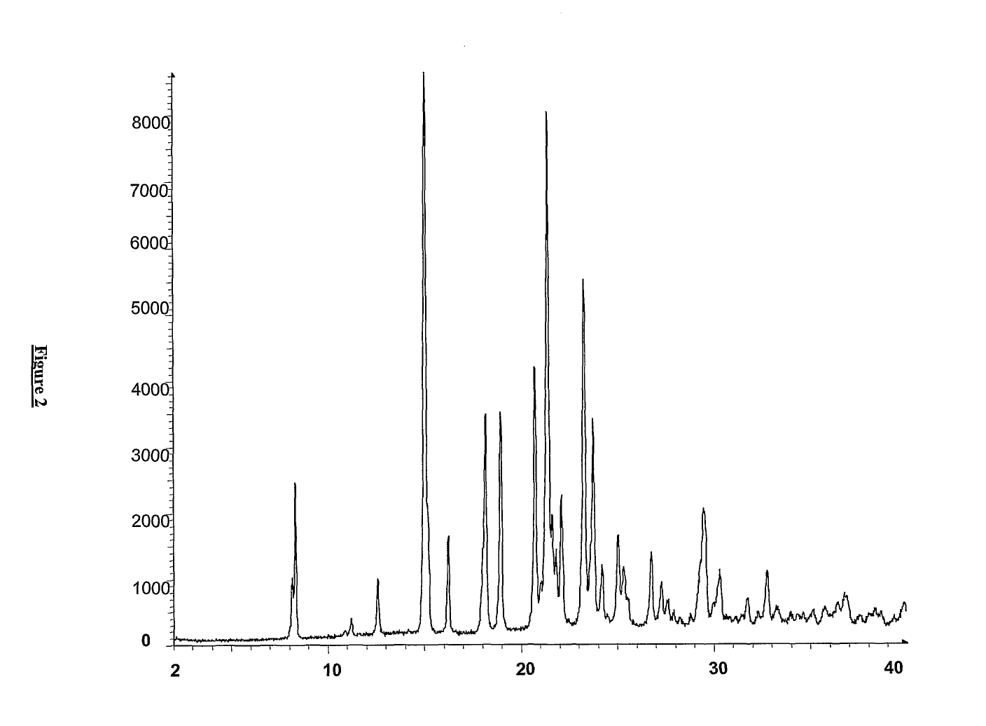

FIG. 3: DVS Isotherm Plot for ZD6474 anhydrous at 25° C.—with target relative humidity (%) on the horizontal axis and change in mass (%) on the vertical axis, wherein the diamonds represent Cycle 1 Sorp, the squares represent Cycle 1 Desporp, the triangles represent Cycle 2 Sorp and the squares represent Cycle 2 Desorp.

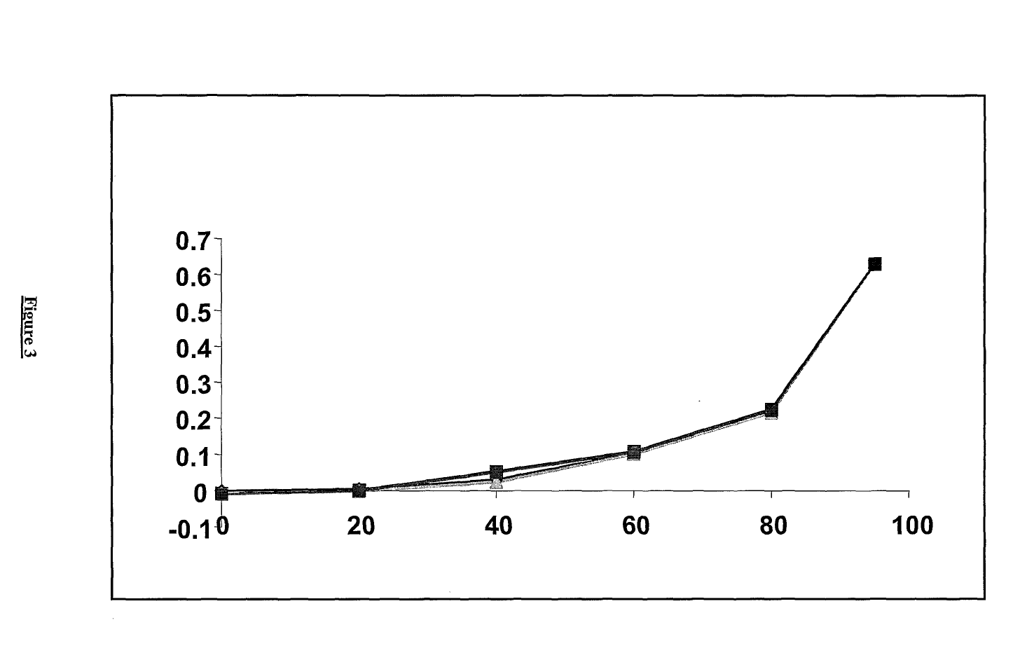

FIG. 4: X-Ray Powder Diffraction Pattern for ZD6474 monohydrate—with the 2 theta values plotted on the horizontal axis and the relative line intensity (counts) plotted on the vertical axis.

FIG. 5: DSC and TGA Thermograms for ZD6474 monohydrate—with temperature in ° C. plotted on the horizontal axis and heat flow/% weight loss on the vertical axis. The top plot is the TGA plot and the lower plot is the DSC plot. The scale on the y axis for the TGA plot is 2 mg as indicated on the graph and the scale on the y axis for the DSC plot is 10 mW as indicated on the graph.

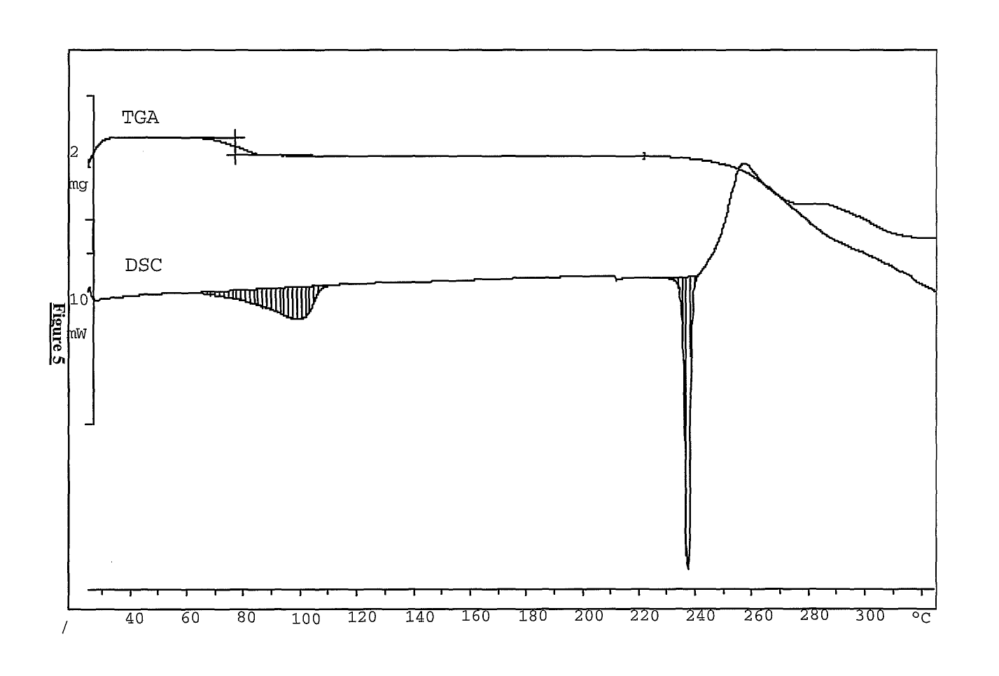

FIG. 6: DVS Isotherm Plot for ZD6474 monohydrate at 25° C.—with target relative humidity (%) on the horizontal axis and change in mass (%) on the vertical axis, wherein the diamonds represent Cycle 1 Sorp, the squares represent Cycle 1 Desporp, the triangles represent Cycle 2 Sorp and the squares represent Cycle 2 Desorp.

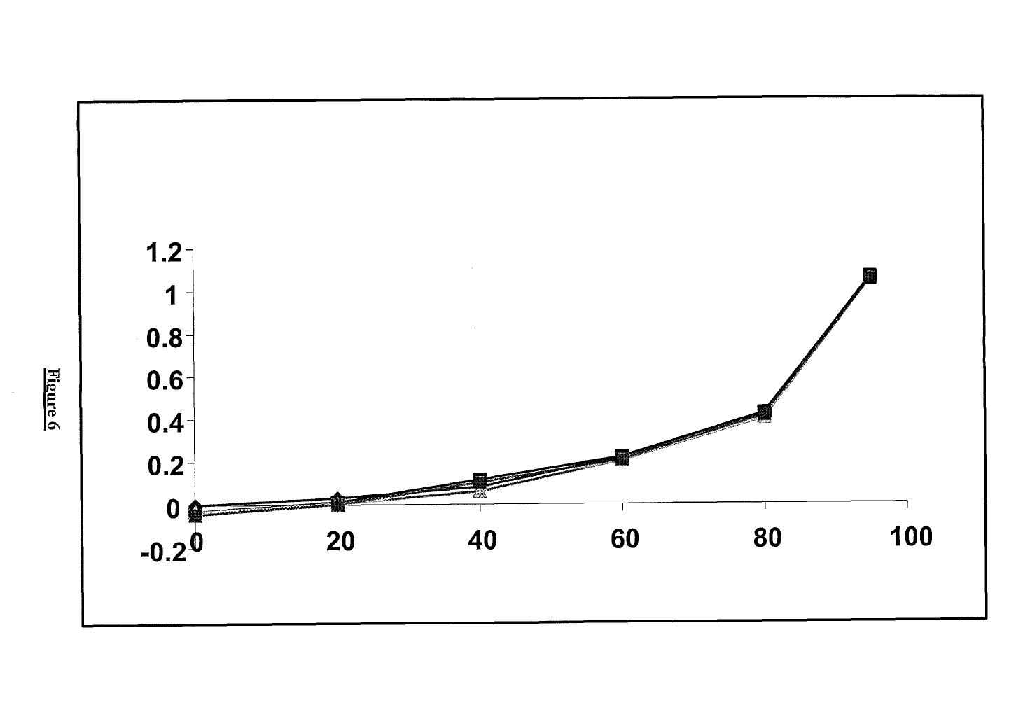

FIG. 7: DVS Isotherm Plot for ZD6474 monohydrate at 0% relative humidity and 25° C.—with time in minutes on the horizontal axis and change in mass (% of initial weight) on the vertical axis.

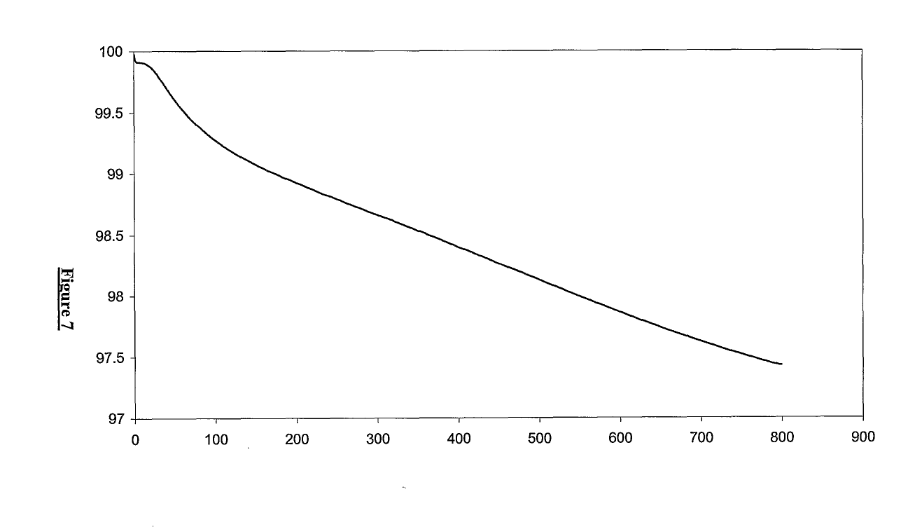

FIG. 8: DVS Isotherm Plot for ZD6474 monohydrate at 0% relative humidity and 40° C.—with time in minutes on the horizontal axis and change in mass (% of initial weight) on the vertical axis.

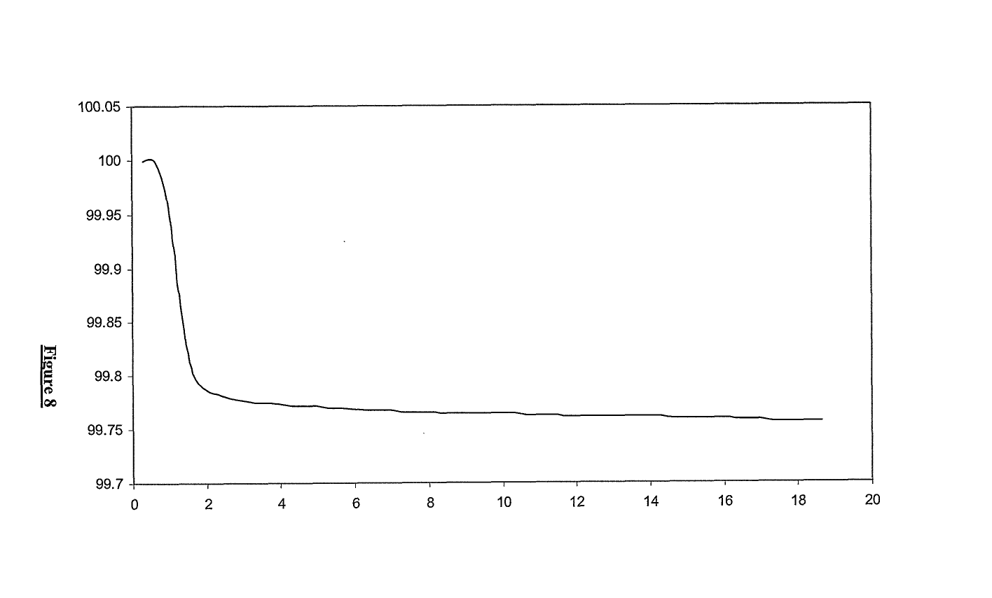

FIG. 9: X-Ray Powder Diffraction Pattern for ZD6474 anhydrous formed in Example 1 of the present application—with the 2 theta values plotted on the horizontal axis and the relative line intensity (counts) plotted on the vertical axis.

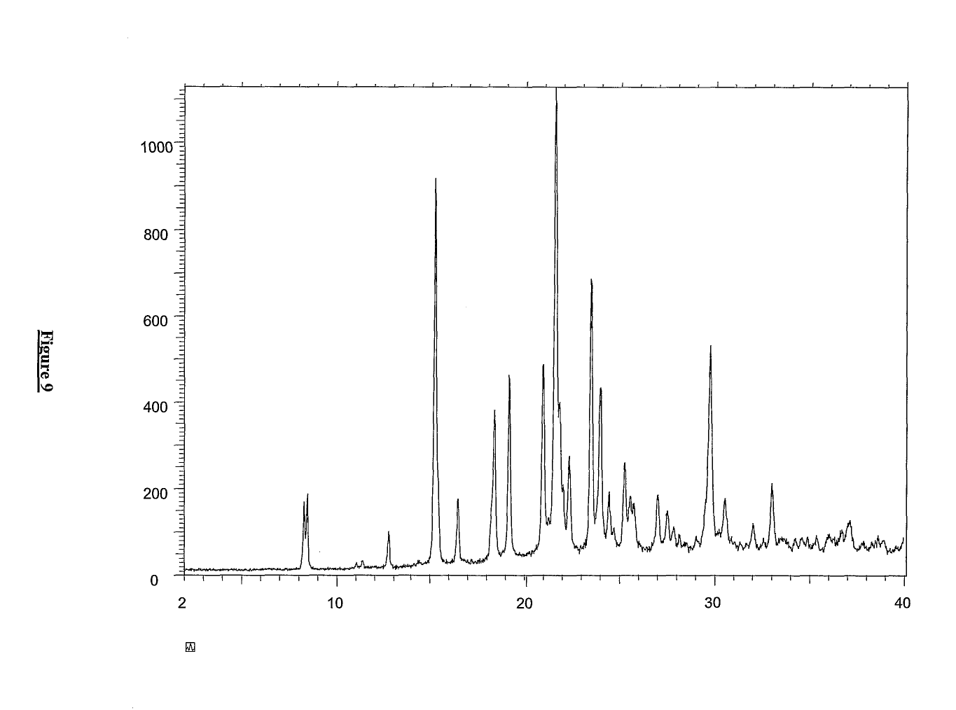

### DETAILS OF TECHNIQUES USED

### X-Ray Powder Diffraction

TABLE 3

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits
Analytical Instrument: Siemens D5000, calibrated using quartz.

The X-ray powder diffraction spectra were determined by mounting a sample of the crystalline ZD6474 material on Siemens single silicon crystal (SSC) wafer mounts and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA using CuKa radiation with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+ software. Persons skilled in the art of X-ray powder diffraction will realise that the relative intensity of peaks can be affected by, for example, grains above 30 microns in size and non-unitary aspect ratios which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

### Dynamic Vapour Sorption

Analytical Instrument: Surface Measurements Systems Dynamic Vapour Sorption Analyser, calibrated with a saturated salt solution, such as sodium chloride.

About 5 mg of material contained in a quartz holder at a specified temperature was subjected to humidified nitrogen at a flow rate of 200 ml/minute of nitrogen at the following relative humidities (RH): 0, 20, 40, 60, 80, 95, 80, 60, 40, 20, 0% RH in duplicate.

The weight of the material at a particular relative humidity was constantly monitored using an in-situ balance until it was stable according to a weight criteria of 0.002% weight change per minute averaged over 10 minutes. If the weight was still changing then it stayed at a particular relative humidity until the weight was stable (up to a maximum time of 12 hours).

### Differential Scanning Calorimetry (DSC)

### Analytical Instrument: Mettler DSC820e.

DSC was conducted by heat reflux DSC using indium metal as a standard calibration. Typically less than 5 mg of material contained in a 40 μl aluminium pan fitted with a pierced lid was heated over the temperature range 25° C. to 325° C. at a constant heating rate of 10° C. per minute. A purge gas using nitrogen was used—flow rate 100 ml per minute. For further information on DSC the reader is referred to: DSC/TGA Instrumental analysis 1986 Christian & O'Reilly, Published by Allyn and Bacon ISBN00205086853,

### Thermogravimetric Analysis (TGA)

Analytical Instrument: Mettler TG851 calibrated for weight using a standard calibration weight.

Typically between 3 and 12 mg of material contained in a 70 μl alox (aluminium oxide) crucible was heated over the temperature range 25° C. to 325° C. at a constant heating rate of 10° C. per minute, whilst constantly monitoring the weight using an in-situ balance. A purge gas using helium was used—flow rate 50 ml per minute.

For further information on TGA the reader is referred to: DSC/TGA Instrumental analysis (1986) Christian & O'Reilly, Published by Allyn and Bacon ISBN00205086853,

### Karl Fischer Water Content

### Analytical Instrument: Mitsubishi Moisture Meter CA-05.

Typically approximately 50 mg of material was used.

For further information on measurement of Karl Fischer Water Content the reader is referred to: Fundamentals of Analytical Chemistry (1996) by Skoog, West and Holler published by Brooks/Cole ISBN0-03-005938-0

1. A 4-(4-bromo-2-fluoroanilino)-6-methoxy-7-(1-methylpiperidin-4-ylmethoxy)quinazoline monohydrate (hereinafter ZD6474 monohydrate).

2. The ZD6474 monohydrate, according to claim 1, in a crystalline form, wherein the monohydrate has an X-ray powder diffraction pattern with at least one specific peak at about 2-theta=10.8°.

3. The ZD6474 monohydrate, according to claim 1, in a crystalline form, wherein the monohydrate has an X-ray powder diffraction pattern with at least one specific peak at about 2-theta=21.0°.

4. The ZD6474 monohydrate, according to claim 1, in a crystalline form, wherein the monohydrate has an X-ray powder diffraction pattern with at least two specific peaks at about 2-theta=10.8 and 21.0°.

5. The ZD6474 monohydrate, according to claim 1, in a crystalline form, wherein the monohydrate has an X-ray powder diffraction pattern with specific peaks at about 2-theta=10.8, 21.0, 18.4, 11.9, 18.9, 18.1, 22.1, 11.4, 20.1 and 24.0°.

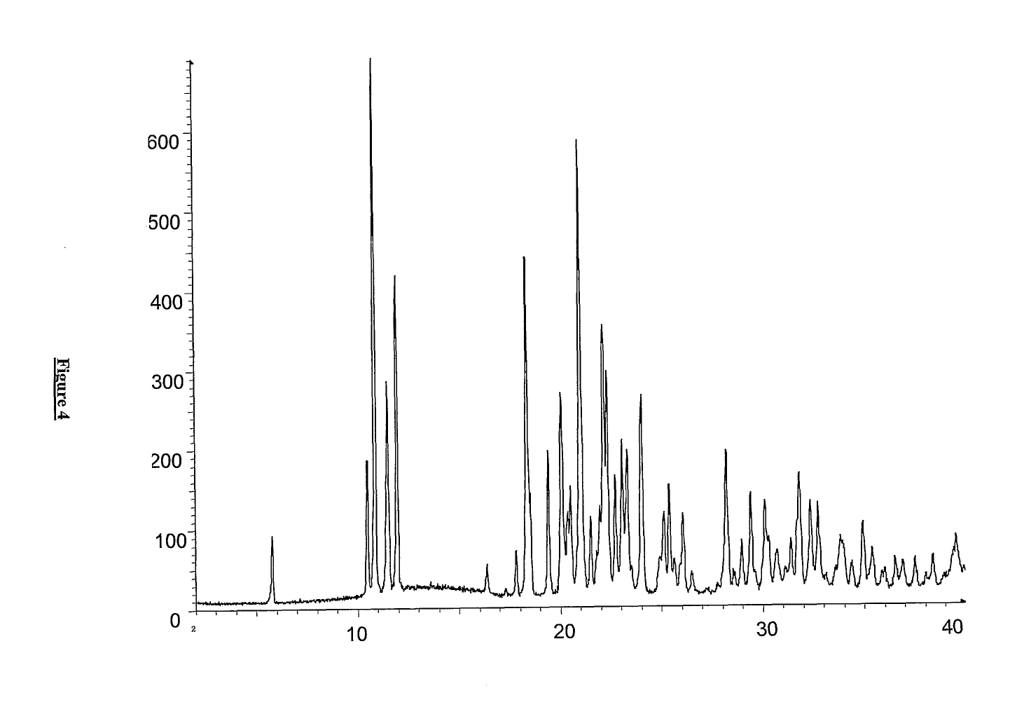

6. The ZD6474 monohydrate, according to claim 1, in a crystalline form, wherein the monohydrate has an X-ray powder diffraction pattern substantially the same as the X-ray powder diffraction pattern shown in FIG. 4.

7. A pharmaceutical composition which comprises a ZD6474 monohydrate according to claim 1 in association with a pharmaceutically acceptable excipient or carrier.

8. A process for the preparation of a ZD6474 monohydrate in the crystalline form as claimed in claim 1, which comprises:
(i) dissolving ZD6474 free base in an aqueous organic solvent mixture to form a solution;
(ii) allowing spontaneous crystallisation to occur; and
(iii) isolating the crystalline solid so formed.

9. A process for the preparation of a ZD6474 monohydrate in the crystalline form as claimed in claim 8, wherein the aqueous organic solvent mixture comprises 90% (by volume) tetrahydrofuran and 10% (by volume) water.

10. (canceled)

11. A method for producing an antiangiogenic and/or vascular permeability reducing effect in a warm-blooded animal in need of such treatment which comprises administering to said animal an effective amount of a ZD6474 monohydrate as claimed in claim 1.
